(19) United States
(12) Patent Application Publication
(10) Pub. No.: US 2003/0119901 A1
(43) Pub. Date: Jun. 26, 2003

(54) Pharmaceutical formulation containing an LTB4 antagonist

(75) Inventors: Robert Becker, Biberach (DE); Thomas Bock, Biberach (DE); Siglinde Moll, Aepfingen Gde. Maselheim (DE); Rene Roscher, Konstanz (DE); Ulrich Roth, Biberach (DE); Achim Sauer, Ravensburg-Torkenweiler (DE); Karl Weber, Grodt/Ingoldingen (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma KG, Ingelheim (DE)

(21) Appl. No.: 10/192,089

(22) Filed: Jul. 10, 2002

Related U.S. Application Data
(60) Provisional application No. 60/315,322, filed on Aug. 28, 2001.

(30) Foreign Application Priority Data
Jul. 14, 2001 (DE) DE 101 34 377.9
Feb. 15, 2002 (DE) DE 102 06 241.2

(51) Int. Cl. A61K 31/155; A61K 31/325; A61K 9/20

(52) U.S. Cl. 514/485; 514/637; 424/465

(57) ABSTRACT

The invention relates to a new pharmaceutical formulation containing an LTB₄ antagonist of formula I

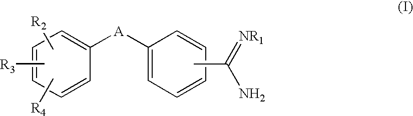

wherein A, R₁, R₂, R₃ and R₄ have the meanings as described herein, as well as the use thereof as pharmaceutical compositions for the treatment of disease.

### RELATED APPLICATIONS

Benefit of U.S. Provisional Application Serial No. 60/315,322, filed on Aug. 28, 2001 is hereby claimed, and said application is herein incorporated by reference in its entirety.

### FIELD OF THE INVENTION

The invention relates to a new pharmaceutical formulation containing an LTB₄ antagonist which has a benzamidine group, processes for the preparation thereof as well as the use thereof as a pharmaceutical composition for the treatment of disease.

### BACKGROUND OF THE INVENTION

LTB₄ antagonists which contain a benzamidine group are compounds with pharmacologically valuable properties. LTB₄ antagonists may provide great therapeutic benefit, for example, in the treatment of treat arthritis, asthma, chronic obstructive lung diseases, psoriasis, ulcerative colitis, Alzheimer's disease, shock, reperfusion damage/ischaemia, cystic fibrosis, atherosclerosis and multiple sclerosis.

Such compounds are known e.g. from International Patent Applications WO 93/16036, WO 94/11341, WO 96/02497, WO 97/21670, WO 98/11062, WO 98/11119, WO 01/25186 and PCT/EP01/00262.

These compounds have the chemical structure of formula I:

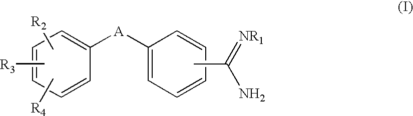

wherein

A denotes a group of formula

—O—CₘH₂ₘ—O—(PHE)ₙ— (II)

wherein

m is an integer from 2 to 6, preferably 2 to 5,

n is 0 or 1,

PHE denotes a 1,4-phenylene group optionally substituted by one or two C₁-C₆ alkyl groups, preferably a 1,4-phenylene group substituted by a C₂-C₄ alkyl group in the ortho position linked to the oxygen;

or

A denotes a group of formula

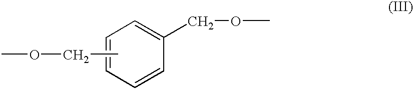

preferably of formula

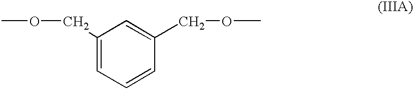

R₁ denotes H, OH, CN, COOR₁₀, or CHO, preferably H or COOR₁₀;

R₂ denotes H, Br, Cl, F, CF₃, CHF₂, OH, HSO₃—O, C₁-C₆-alkyl, C₁-C₆-alkoxy, C₅-C₇-cycloalkyl, CONR₈R₉, aryl, O-aryl, CH₂-aryl, CR₅R₆-aryl, or C(CH₃)₂—R₇, preferably OH, HSO₃—O, CONR₈R₉ or CR₅R₆-aryl,

R₃ denotes H, C₁-C₆-alkyl, C₁-C₆-alkoxy, OH, Cl or F, preferably H or C₁-C₃-alkoxy,

R₄ denotes H or C₁-C₆-alkyl, preferably H;

R₅ denotes C₁-C₄-alkyl, CF₃, CH₂OH, COOH or COO(C₁-C₄-alkyl), preferably C₁-C₄-alkyl, particularly methyl;

R₆ denotes H, C₁-C₄-alkyl or CF₃, preferably C₁-C₄-alkyl, particularly methyl;

R₇ denotes CH₂OH, COOH, COO(C₁-C₄-alkyl), CONR₈R₉ or CH₂NR₈R₉;

R₈ denotes H, C₁-C₆-alkyl, phenyl, phenyl-(C₁-C₆-alkyl), COR₁₀, COOR₁₀, CHO, CONH₂, CONHR₁₀, SO₂—(C₁-C₆-alkyl), SO₂-phenyl, while the phenyl group may be mono- or disubstituted by Cl, F, CF₃, C₁-C₄-alkyl, OH and/or C₁-C₄-alkoxy, and preferably denotes C₁-C₄-alkyl, particularly isopropyl;

R₉ denotes H or C₁-C₆-alkyl, preferably H or C₁-C₄-alkyl, particularly isopropyl; or

R₈ and R₉ taken together represent a C₄-C₆-alkylene group;

R₁₀ denotes C₁-C₆-alkyl, C₅-C₇-cycloalkyl, aryl, heteroaryl, aralkyl or heteroaryl-(C₁-C₆-alkyl), preferably C₁-C₄-alkyl,

while the aryl groups mentioned in groups R₂ and R₁₀ denote phenyl or naphthyl, the heteroaryl groups denote pyrrole, pyrazole, imidazole, furanyl, thienyl, pyridine or pyrimidine and may each be mono- or polysubstituted by Cl, F, CF₃, C₁-C₄-alkyl, OH, HSO₃—O or C₁-C₄-alkoxy, preferably by OH or HSO₃—O—.

### SUMMARY OF THE INVENTION

The aim of the present invention is to provide an orally administered pharmaceutical formulation which releases an active substance of formula I relatively fast and completely and thus leads to greater bioavailability of this active substance. A further object of the present invention is to prepare a formulation which is characterised by ease of handling during the preparation process and thus can be produced industrially in a reproducible manner while maintaining a constant high quality.

### BRIEF DESCRIPTION OF THE DRAWINGS

FIG. 1 shows the blood plasma concentration of the compound of formula IA1 versus time upon administration of three different tablet formulations according to the invention.

FIG. 2 is a flow diagram depicting a direct compression method for manufacturing an example of a tablet formulation according to the invention.

### DETAILED DESCRIPTION OF THE INVENTION

The objective stated above are solved by the formulation described in detail hereinafter.

The present invention relates to a tablet containing a compound of formula I which comprises a compound of formula I, or a pharmacologically acceptable acid addition salt thereof or glucuronide thereof, and at least one pharmacologically acceptable excipient, the tablet comprising at least one wetting agent.

A further aim is the use of a tablet according to invention for preparing a pharmaceutical composition with increased bioavailability for the treatment or prevention of diseases in which LTB₄ antagonists can be used therapeutically or preventively.

The active substance of formula I may be present in the formulation according to the invention in the form of a physiologically acceptable acid addition salt. By physiologically acceptable acid addition salts are meant, according to the invention, pharmaceutically acceptable salts which are selected from the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid. Mixtures of the above acids may also be used to prepare the salts. According to the invention, the preferred salts of formula I are selected from among the hydrochloride, hydrobromide, sulphate, phosphate, fumarate and methanesulphonate. The salts selected from among the hydrochloride, hydrobromide and fumarate are particularly preferred. The active substance may optionally be in the form of a hydrate. Preferably, according to the invention, the compound of formula I is added to the tablet in the form of the free base and in the anhydrous form.

Particularly preferred compounds of formula I are the compounds of formulae IA, IB and IC, particularly IA:

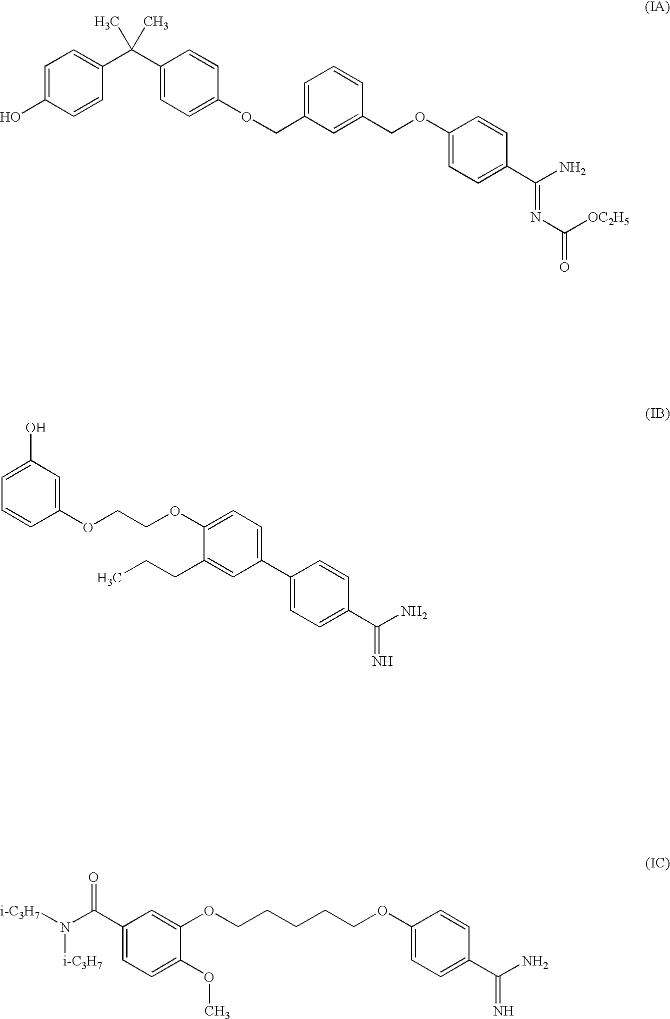

The compounds of formula I wherein R₁ is different from hydrogen are generally prodrugs which are converted in vivo into the corresponding compounds of formula I wherein R₁ is hydrogen.

For example, from the compound IA is formed in vivo the compound of formula IA1:

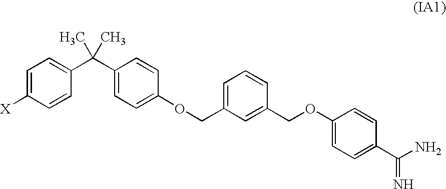

wherein X denotes OH, HSO₃—O or a carbohydrate group of formula C₆H₁₁O₅—O—.

Preferably, the active substance is used in crystalline, unground form or in ground form, particularly in jet-ground form, wherein the particle size distribution is within the following limits: D₁₀≦3 μm, D₅₀ 3 to 8 μm, D₉₀≦8 to 30 μm. The abovementioned numerical data for D₁₀, D₅₀ and D₉₀ in μm (microns) are the particle size ranges within which a throughput total of 10 vol. %, 50 vol. % or 90 vol. % of the particles measured (cumulative volume distribution) is achieved. These values were determined by the laser diffractometry method, specifically, in the present instance, using a so-called dry dispersion under a dispersion pressure of 2 bar and with a focal length f=500 mm, e.g. using a Sympatec/RODOS apparatus. This methodology is known in the prior art.

Where reference is made to salts of the compounds of formula I within the scope of the present invention, this is indicated by the symbol I′. Explicit references to the free base of formula I, on the other hand, are indicated by the use of the symbol I.

In relation to the total mass of the tablets according to the invention the compound of formula I, particularly IA is present in an amount of up to 0.2 to 80 wt. %, preferably 0.7 to 40 wt. %, more preferably about 5 to 38 wt. %. Particularly preferably, the content of the free base of I is between 6 and 36 wt. % based on the total mass of the tablet.

The term “wetting agent” as used hereinbefore and hereinafter denotes an excipient which lowers the surface tension of water or other liquids so that they can penetrate into the surfaces of the tablets according to the invention and soak through them, displacing the air, thereby wetting them. The substances used as wetting agents are usually interface-active surfactants. These surfactants are amphiphilic (bifunctional) compounds with at least one hydrophobic and one hydrophilic part of the molecule. The hydrophobic group is usually a hydrocarbon chain, if possible a straight chain, with eight to 22 carbon atoms. Particular surfactants may also have (dimethyl)-siloxane chains or perfluorinated hydrocarbon chains as the hydrophobic part of the molecule. The hydrophilic group is either a negatively or positively charged (hydratable) or a neutral polar head group. Of the surfactants, anionic surfactants, particularly the long-chain alkylsulphates, especially sodium laurylsulphate and alkylbenzenesulphonates are preferred.

According to the invention, the weight ratio between the components, namely wetting agent and active substance I, contained in the tablet is in a range from about 1:200 to about 1:5, preferably between about 1:150 and about 1:10.

The pharmaceutical formulation according to the invention contains in addition to the active substance at least one excipient as a filler/dry binder.

Within the scope of the present invention carbohydrates such as lactose or mannose, particularly finely divided lactose or sugar alcohols such as mannitol, sorbitol or xylitol, particularly mannitol, are of particular importance as excipients. These excipients have proved particularly advantageous in the formulation according to the invention. In a preferred aspect, therefore, the present invention relates to a tablet containing at least one compound of formula I, which contains, in addition to the active substance and the wetting agent, lactose, particularly finely divided lactose, more preferably lactose monohydrate or mannitol as excipient.

According to the invention the weight ratio between the components lactose or mannitol to active substance I contained in the tablet is in a range from about 20:1 to about 1:4. Preferably, the ratio of lactose to I is in a range from about 1.8:1 to about 1:1.2, more preferably in a range from about 1.4:1 to 1.6:1. Preferably, the proportion by weight of lactose based on the total mass of the tablet according to the invention is in a range of about 1-70 wt. %, preferably about 2-60 wt. %.

Preferably, the ratio of mannitol to I is in a range of about 20:1 to about 1:2, more preferably in a range of about 15:1 to 3:1. Preferably the proportion by weight of mannitol based on the total mass of the tablet according to the invention is in a range of about 20-90 wt. %, preferably between about 40-75 wt. %.

The tablet according to the invention may also contain, in addition to lactose, wetting agent and active substance, further excipients or fillers. According to the invention, it is preferable to use compounds capable of acting as binders.

The term “binder” used hereinbefore and hereinafter denotes excipients which are suitable for binding other components to one another. Preferred binders according to the invention are selected from among:

powdered cellulose, microcrystalline cellulose, sorbitol, starch, polyvinylpyrrolidone (povidone), copolymers of vinylpyrrolidone with other vinyl derivatives (copovidone), cellulose derivatives, particularly methylhydroxypropylcellulose, e.g. Methocel A 15 LV, and mixtures of these compounds. The preferred binders are powdered cellulose, particularly microcrystalline cellulose and/or copovidone. Most preferred is a mixture of microcrystalline cellulose and a copolymer of vinylpyrrolidone and vinyl acetate, namely copovidone VA 64, the ratio of vinylpyrrolidone and vinyl acetate being about 3:2 (m/m). As a rule the tablet according to the invention has a weight ratio of microcrystalline cellulose to copovidone VA 64 of 20:1 to 1:1, preferably 15:1 to 2:1, particularly about 10:1 to 3:1. Thanks to this particularly preferred binder combination of microcrystalline cellulose and copovidone, tablets are obtained having a high bioavailability of the compounds of formula I.

If one of the abovementioned binders is added to the formulation according to the invention, the weight ratio of lactose to binder is preferably about 10:1 to about 1:2, preferably about 5:1 to about 1:1, more preferably about 4.5:1 to 3.5:2.

If one of the abovementioned binders is added to the formulation according to the invention, the weight ratio of mannitol to binder is preferably about 10:1 to about 1:2, preferably about 5:1 to about 1:1, more preferably about 3.5:1 to 3.0:1.

The tablet according to the invention may also contain disintegrants in addition to the abovementioned ingredients. Within the scope of the present invention these disintegrants may optionally also be known as breakdown agents. These are preferably selected, according to the invention, from among sodium starch glycolate, crosslinked polyvinylpyrrolidone (crospovidone), croscarmellose sodium salt (sodium salt of cellulose carboxymethyl ether, crosslinked), sodium-carboxymethylcellulose, dried maize starch and mixtures thereof. Within the scope of the present invention it is particularly preferred to use sodium starch glycolate, crospovidone and, preferably, the sodium salt of crospovidone or croscarmellose. If the abovementioned disintegrants are used, the amount by weight, based on the total mass of the tablet according to the invention, is preferably in a range of about 0.5-10 wt. %, more preferably about 1.5-7.5 wt. %.

The tablet according to the invention may also contain flow agents or flow regulators and also lubricants, as additional ingredients. These include, within the scope of the present invention, for example, silicon dioxide, talc, stearic acid, sodium stearylfumarate, magnesium stearate and glycerol tribehenate. According to the invention magnesium stearate is preferably used. If the abovementioned flow agents or flow regulators or lubricants are used, the amount by weight thereof, based on the total mass of the tablet according to the invention, is preferably in a range of about 0.1-10 wt. %, preferably about 0.5-5 wt. %, more preferably between 0.6 and 1.5 wt. %.

In addition, the tablet according to the invention may contain one or more synthetic or natural, pharmaceutically acceptable dyes or colourings, preferably indigo carmine. If the abovementioned colourings are used the amount by weight thereof based on the total mass of the tablet according to the invention is 0.01 to 0.5 wt. %.

Particularly preferred is a tablet consisting essentially of the following ingredients:

a compound of formula I, preferably 5 wt. % to 50 wt. %, particularly 6 wt. % to 45 wt. % of a compound of formula IA;

microcrystalline cellulose, preferably 0., wt. % to 25 wt. %, particularly 5 wt. % to 20 wt. % microcrystalline cellulose;

lactose, preferably 15 wt. % to 80 wt. %, particularly 20 wt. % to 75 wt. % lactose; or mannitol, preferably 20 wt. % to 90 wt. %, particularly 40 wt. % to 75 wt. % mannitol;

optionally a copolymer of vinylpyrrolidone and vinyl acetate, preferably 0.2 wt. % to 5 wt. %, particularly 0.5 wt. % to 2.5 wt. % of copovidone V64,

sodium lauryl sulphate, preferably 0.01 wt. % to 10 wt. %, particularly 0.06 wt. % to 7 wt. % of sodium lauryl sulphate;

crosslinked polyvinylpyrrolidone, preferably 0.4 wt. % to 8 wt. %, particularly 0.8 wt. % to 6 wt. %; or crosslinked cellulose carboxymethylether sodium salt, particularly croscarmellose sodium salt;

magnesium stearate, preferably 0.1 wt. % to 5 wt. %, particularly 0.5 wt. % to 1.5 wt. % of magnesium stearate; and

optionally a colouring.

The tablet according to the invention may be prepared by directly mixing and compressing the ingredients or by granulation and compression. To prepare the tablet according to the invention the following procedure may be used, for example.

The active substance of formula I, the wetting agent and optionally a copolymer of vinylpyrrolidone and vinyl acetate as binder are dissolved or suspended in water, granulated with the excipients, particularly lactose monohydrate and optionally microcrystalline cellulose as an additional binder, screened and then dried. The product obtained is optionally mixed with the flow agent, particularly magnesium stearate and screened. Then a disintegrant, particularly crospovidone, is optionally added. The mixture of active substance and excipient thus obtained is then compressed in a suitable tablet press to form the tablets according to the invention.

The compression forces which are needed to produce tablets of suitable breaking resistance and hence with the required breakdown times are dependent on the shapes and sizes of the punching tools used. Compression forces in the range from 2-20 kN are preferred. Higher compression forces may lead to tablets with a delayed released of active substance. Lower compression forces may produce mechanically unstable tablets. The tablet cores may have different shapes; the preferred shapes are round biplanar or biconvex and oval or oblong forms.

The Examples that follow serve to illustrate some formulations according to the invention. They are intended solely as possible procedures described by way of example, without restricting the invention to their content.

### EXAMPLE 1

| Ingredients | mg/tablet | volatile part |
|---|---|---|
| (01) compound IA, jet-ground | 75,000 | |
| (02) microcrystalline cellulose | 22,500 | |
| (03) lactose | 114,560 | |
| (04) copovidone VA 64 | 3,750 | |
| (05) sodium laurylsulphate | 0,750 | |
| (06) water | | 39,000 |
| (07) cropovidone | 6,750 | |
| (08) magnesium stearate | 1,690 | |
| | 225,000 | 39,000 |

Preparation (Variant 1)

1 batch=2992.5 g=13,300 tablets

1. Premix

In a suitable intensive mixer, e.g. Diosna P10, 997.500 g of jet-ground compound of formula IA, 299.250 g of microcrystalline cellulose and 1523.648 g of lactose monohydrate are mixed for 3 minutes.

2. Granulating Liquid

518,700 g of water are placed in a suitable container. 49,875 g of copovidone VA 64 are slowly stirred in using a suitable stirrer. After the povidone VA 64 has dissolved, 9.975 g of sodium laurylsulphate are stirred in.

3. Granules

After the addition of the granulating liquid 2. using a liquid funnel the premix 1. is mixed wet and granulated in an intensive mixer.

Moist Screening

The moist granules thus produced are moist screened with a suitable screening machine, e.g. Erweka AR401, with a mesh size of 1.6 mm.

Drying

In a suitable drying cupboard, e.g. a WTB Binder, the granules are dried for 10 hours in a layer thickness of about 1 cm at a temperature of 50° C.

Dry Screening

Using a suitable screening machine, e.g. Erweka AR401, the dried granules are dry-screened with a mesh size of 0.8 mm.

4. Final Mixture

In a suitable container with a suitable mixer, e.g. an Rm 100 gyrowheel mixer, 2880.248 g of the screened granules 3. are added to 89.775 g of crospovidone at 30 rpm and the components are homogeneously mixed for 10 minutes. Then 22.477 g of magnesium stearate are added to the mixture and it is homogeneously mixed for 5 minutes.

5. Tablet Making

In a suitable tablet press, e.g. EK 0 (pressing tool: 9 mm with radius of convexity of 13.5 mm and faceted), 2992.500 g of the final mixture 4. are compressed at a compression speed of 25 tablets./min. to produce tablets weighing 225 mg each.

The tablets thus obtained have the following properties:

| weight | 225 mg |
|---|---|
| diameter | 9 mm |
| height | about 3.6 mm |
| breaking strength | 100 N |
| | (n = 10 hrs) |
| disintegration | ≦8 minutes |
| friability | |
| - abrasion | <0.5% |

### EXAMPLE 2

| Ingredients | mg/tablet | volatile part |
|---|---|---|
| (01) compound IA, jet-ground | 75,000 | |
| (02) microcrystalline cellulose | 22,500 | |
| (03) fine lactose | 114,560 | |
| (04) copovidone VA 64 | 3,750 | |
| (05) sodium laurysulphate | 7,500 | |
| (06) water | | 39,000 |
| (07) crospovidone | 6,750 | |
| (08) magnesium stearate | 1,740 | |
| | 231,800 | 39,000 |

The tablets are prepared analogously to Example 1. The tablets obtained have comparable physical data.

### EXAMPLE 3

| Ingredients | mg/tablet | volatile part |
|---|---|---|
| (01) compound IA, jet-ground | 75,000 | |
| (02) microcrystalline cellulose | 68,250 | |
| (03) fine lactose | 68,500 | |
| (04) Methocel A 15 LV | 3,750 | |
| (05) sodium laurylsulphate | 0,750 | |
| (06) water | | 39,000 |
| (07) cropovidone | 6,750 | |
| (08) magnesium stearate | 2,250 | |
| | 225,000 | 39,000 |

The tablets are prepared analogously to Example 1. The tablets obtained have comparable physical data, but lower hardness and a slower rate of dissolution.

### EXAMPLE 4

| Ingredients | mg/tablet | volatile part |
|---|---|---|
| (01) compound IB, jet-ground | 75,000 | |
| (02) microcrystalline cellulose | 22,500 | |
| (03) fine lactose | 114,560 | |
| (04) copoivdone VA 64 | 3,750 | |
| (05) sodium laurylsulphate | 0,750 | |
| (06) water | | 39,000 |
| (07) crospovidone | 6,750 | |
| (08) magnesium stearate | 1,690 | |
| | 225,000 | 39,000 |

The tablets are prepared analogously to Example 1. The tablets obtained have comparable physical data.

### EXAMPLE 5

| Ingredients | mg/tablet | volatile part |
|---|---|---|
| (01) compound IC, jet-ground | 75,000 | |
| (02) microcrystalline cellulose | 22,500 | |
| (03) fine lactose | 114,560 | |
| (04) copovidone VA 64 | 3,750 | |
| (05) sodium laurylsulphate | 0,750 | |
| (06) water | | 39,000 |
| (07) crospovidone | 6,750 | |
| (08) magnesium stearate | 1,690 | |
| | 225,000 | 39,000 |

The tablets are prepared analogously to Example 1. The tablets obtained have comparable physical data.

### EXAMPLE 6

| Ingredients | mg/tablet | volatile part |
|---|---|---|
| (01) compound IA, jet-ground | 75,000 | |
| (02) microcrystalline cellulose | 22,500 | |
| (03) lactose | 114,560 | |
| (04) copovidone VA 64 | 3,750 | |
| (05) sodium laurylsulphate | 0,750 | |
| (06) water | | 39,000 |
| (07) crospovidone | 6,750 | |
| (08) magnesium stearate | 1,690 | |
| | 225,000 | 39,000 |

Preparation (Variant 2)

1. Premix

In a suitable one-pot granulator, e.g. Zanchetta Roto P 50, 4.6500 kg of jet-ground compound of formula IA, 1.3950 kg of microcrystalline cellulose, 7.1028 kg lactose and 0.4185 kg crospovidone are mixed for 3 minutes.

2. Granulating Liquid

2.1762 kg of water (corresponding to 90% of the quantity in process 1) are placed in a suitable container. 0.2325 kg of copovidone VA 64 are slowly stirred in using a suitable stirrer. After the povidone VA 64 has dissolved, 0.0465 kg of sodium laurylsulphate are stirred in.

3. Granules

After the addition of the granulating liquid the premix 1. is mixed wet and granulated in a one-pot granulator.

Drying

The granules are dried for 50 minutes in a one-pot granulator at a temperature of 49° C.

Dry Screening

Using a suitable screening machine, e.g. Comil 197 S, the dried granules are dry-screened with a mesh size of RS 2007.

4. Final Mixture

In a suitable gravity mixer, e.g. Servolift Kubus 60 1, 13,8453 kg of the screened granules 3. and 0.1047 kg magnesium stearate are homogeneously mixed at 10 rpm for 5 minutes.

5. Tablet Making

In a suitable tablet press, e.g. Fette P 1200 (pressing tool: 9 mm with radius of convexity of 13.5 mm and faceted), 13.9500 kg of the final mixture 4. are compressed at a compression speed of 100,000 tablets./hr to produce tablets weighing 225 mg each.

### EXAMPLE 7

| Ingredients | mg/tablet |
|---|---|
| (01) compound IA, jet-ground | 5,000 |
| (02) microcrystalline cellulose | 15,000 |
| (03) mannitol | 52,250 |
| (04) croscarmellose sodium | 1,500 |
| (05) sodium laurylsulphate | 0,050 |
| (06) indigo carmine (11-14%) | 0,075 |
| (07) magnesium stearate | 1,125 |
| | 75,000 |

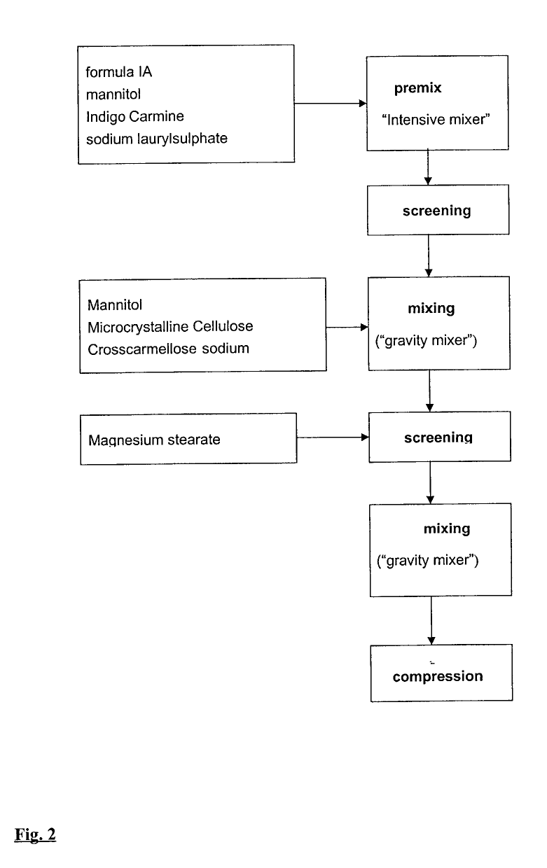

The direct compression comprises producing a premix of ingredients (01), (05), (06) and some of (03) with a Diosna® intensive mixer. The premix is screened and mixed with ingredients (02), (04) and the remainder of (03) in a Turbula® gravity mixer. After the mixture has been screened again, ingredient (07) is added. A corresponding flow diagram is shown in FIG. 2.

Biconvex tablets 6 mm thick with rounded edges, containing 5 mg of the compound of formula IA with a total weight of 75 mg are compressed

### EXAMPLE 8

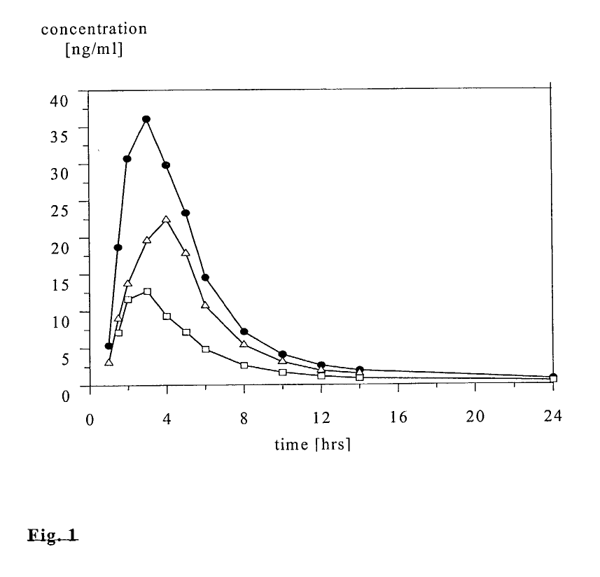

The tablets of Examples 1 to 3 according to the invention are administered to healthy male subjects by oral route in a single dose. The plasma concentration of the active substance is determined from time to time. The mean data thus obtained are shown graphically in FIG. 1. In FIG. 1 the blood plasma concentration in ng/ml of the glycoside of the compound of formula IA1 produced in vivo from the compound of formula IA is plotted against the time in hours. According to this, the highest concentration of active substance in the plasma is obtained with the tablet according to Example 1.

The symbols used in FIG. 1 have the following meanings:

- -: tablet of Example 1

-Δ-: tablet of Example 2

- -: tablet of Example 3

We claim:

1. A pharmaceutical formulation in the form of a tablet comprising an LTB₄ antagonist of formula I: 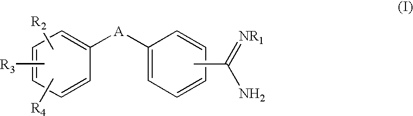
wherein
A denotes a group of formula
—O—CₘH₂ₘ—O—(PHE)ₙ— (II)
wherein
m is an integer from 2 to 6,
n is 0 or 1,
PHE denotes a 1,4-phenylene group optionally substituted by one or two C₁-C₆ alkyl groups;
or
A denotes a group of formula 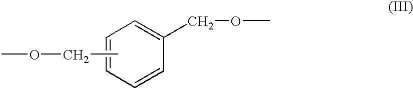
R₁ denotes H, OH, CN, COOR₁₀, or CHO;
R₂ denotes H, Br, Cl, F, CF₃, CHF₂, OH, HO₃—O, C₁-C₆-alkyl, C₁-C₆-alkoxy, C₅-C₇-cycloalkyl, CONR₈R₉, aryl, O-aryl, CH₂-aryl, CR₅R₆-aryl, or C(CH₃)₂-R₇,
R₃ denotes H, C₁-C₆-alkyl, C₁-C₆-alkoxy, OH, Cl or F,
R₄ denotes H or C₁-C₆-alkyl;
R₅ denotes C₁-C₄-alkyl, CF₃, CH₂OH, COOH or COO(C₁-C₄-alkyl);
R₆ denotes H, C₁-C₄-alkyl or CF₃;
R₇ denotes CH₂OH, COOH, COO(C₁-C₄-alkyl), CONR₈R₉ or CH₂NR₈R₉;
R₈ denotes H, C₁-C₆-alkyl, phenyl, phenyl-(C₁-C₆-alkyl), COR₁₀, COOR₁₀, CHO, CONH₂, CONHR₁₀, SO₂-(C₁-C₆-alkyl), or SO₂-phenyl wherein the phenyl group may be mono- or disubstituted by Cl, F, CF₃, C₁-C₄-alkyl, OH and/or C₁-C₄-alkoxy;
R₉ denotes H or C₁-C₆-alkyl; or
R₈ and R₉ taken together represent a C₄-C₆-alkylene group;
R₁₀ denotes C₁-C₆-alkyl, C₅-C₇-cycloalkyl, aryl, heteroaryl, aralkyl or heteroaryl-(C₁-C₆-alkyl),
wherein the aryl groups mentioned in groups R₂ and R₁₀ independently denote phenyl or naphthyl, and the heteroaryl groups independently denote pyrrolyl, pyrazolyl, imidazolyl, furanyl, thienyl, pyridinyl or pyrimidinyl and may each be mono- or polysubstituted by Cl, F, CF₃, C₁-C₄-alkyl, OH, HSO₃—O or C₁-C₄-alkoxy, or a pharmacologically acceptable acid addition salt or glycoside or 0-sulphate thereof; and at least one pharmacologically acceptable excipient, wherein the tablet contains at least one wetting agent.

2. A tablet according to claim 1, wherein the LTB₄ antagonist is a compound selected from among formulas IA, IB and IC:

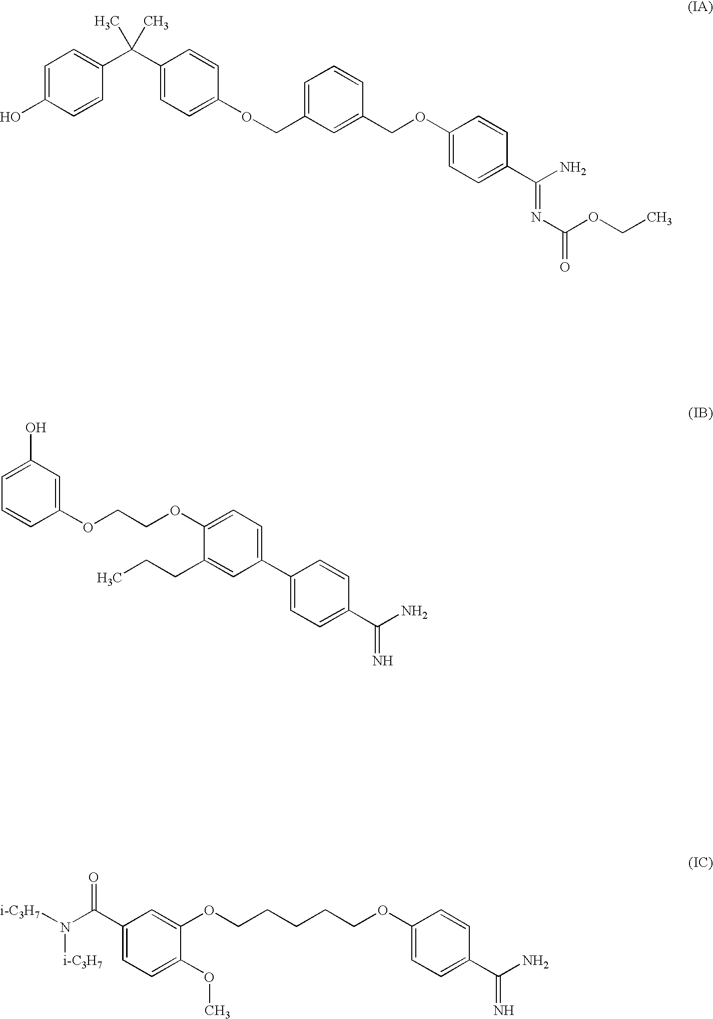

3. A tablet according to claim 1, wherein the wetting agent is an anionic surface-active substance.

4. A tablet according to claim 1, wherein the wetting agent is sodium laurylsulphate.

5. A tablet according to claim 1, wherein the excipient is lactose or mannitol.

6. A tablet according to claim 1, further comprising at least one dry binder, at least one lubricant, at least one disintegrant and/or at least one separating agent and optionally a coloring.

7. A tablet according to claim 6, wherein the dry binder is selected from among powdered cellulose, microcrystalline cellulose, starch, polyvinylpyrrolidone, copolymers of vinylpyrrolidone with other vinyl derivatives, cellulose derivatives and mixtures of these compounds.

8. A tablet according to claim 6, wherein the dry binder is microcrystalline cellulose or a mixture of microcrystalline cellulose and a copolymer of vinylpyrrolidone and vinyl acetate.

9. A tablet according to claim 1, wherein it comprises 0.2 to 80 wt. % of a compound of formula I.

10. A tablet according to claim 1, wherein it comprises 0.7 to 40 wt. % of a compound of formula I.

11. A tablet according to claim 1, wherein the weight ratio between the wetting agent and the active substance of formula I is in a range of about 1:200 to about 1:5.

12. A tablet according to claim 1, wherein the weight ratio between the wetting agent and the active substance of formula I is in a range of about 1:150 to about 1:10.

13. A tablet according to claim 5, wherein the weight ratio between lactose or mannitol and the active substance of formula I is in the range from about 20:1 to about 1:4.

14. A tablet according to claim 5, wherein the weight ratio between lactose or mannitol and the active substance of formula I is in the range from about 12:1 to about 1:1.2.

15. A tablet according to claim 5, wherein the weight ratio between lactose and the active substance of formula I is in the range from about 4:1 to about 1:4.

16. A tablet according to claim 5, wherein the weight ratio between lactose and the active substance of formula I is in the range from about 1.8:1 to about 1:1.2.

17. A tablet according to claim 5, wherein the weight ratio between mannitol and the active substance of formula I is in a range of about 20:1 to about 1:2.

18. A tablet according to claim 5, wherein the weight ratio between mannitol and the active substance of formula I is in a range of about 15:1 to 3:1.

19. A tablet according to claim 6, wherein the amount by weight of the disintegrant based on the total mass of the tablet is in a range from about 0.5 to 10 wt. %.

20. A tablet according to claim 5, further comprising a flow agent, a lubricant and a separating agent, wherein the amount by weight of the flow agent, lubricant and separating agent based on the total mass of the tablet is in a range from about 0.1 to 5 wt. %.

21. A tablet according to claim 1, consisting essentially of the following ingredients:
(a) a compound of formula I;
(b) microcrystalline cellulose;
(c) lactose or mannitol;
(d) optionally a copolymer of vinylpyrrolidone and vinyl acetate;
(e) sodium laurylsulphate;
(f) crosslinked polyvinylpyrrolidone or a crosslinked cellulosecarboxymethylether sodium salt;
(g) magnesium stearate; and
(h) optionally a pharmaceutically acceptable coloring.

22. A tablet according to claim 21, wherein the crosslinked cellulosecarboxymethylether sodium salt is croscarmellose sodium salt;

23. A process for preparing a tablet according to claim 1, comprising the following steps:
(a) mixing a compound of formula I with a wetting agent and optionally other pharmacologically acceptable excipients, optionally in the presence of a volatile diluent;
(b) screening the mixture obtained in step (a);
(c) optionally adding a lubricant to the product of step (b), and
(d) compressing the resulting product with a suitable tablet press.

24. A method for treating a disease in which an LTB₄ antagonist can be used therapeutically to effect treatment, comprising administering to a patient in need thereof an effective amount of a tablet according to claim 1.

25. A method for treating a disease selected from arthritis, asthma, chronic obstructive pulmonary disease, psoriasis, ulcerative colitis, Alzheimer's disease, shock, reperfusion damage/ischaemia, cystic fibrosis, atherosclerosis and multiple sclerosis, comprising administering to a patient in need thereof an effective amount of a tablet according to claim 1.
